(19) United States
(12) Patent Application Publication
(10) Pub. No.: US 2015/0283091 A1
(43) Pub. Date: Oct. 8, 2015

(54) PHARMACEUTICAL COMPOSITIONS COMPRISING HYDROMORPHONE AND NALOXONE

(71) Applicant: PURDUE PHARMA, Pickering (CA)

(72) Inventor: Ricardo Alberto Vargas Rincon, Mississauga (CA)

(21) Appl. No.: 14/441,814

(22) PCT Filed: Nov. 6, 2013
(86) PCT No.: PCT/CA2013/000932

§ 371 Date: May 8, 2015

Related U.S. Application Data
(60) Provisional application No. 61/796,390, filed on Nov. 9, 2012.

(30) Foreign Application Priority Data
Nov. 9, 2012 (CA) 2,795,324

(51) Int. Cl. A61K 9/50; A61K 47/18; A61K 47/26; A61K 9/48; A61K 47/38; A61K 47/32; A61K 31/485; A61K 47/02

(52) U.S. Cl. CPC A61K 9/5078; A61K 31/485; A61K 47/183; A61K 47/02; A61K 9/5047; A61K 9/5031; A61K 9/4808; A61K 9/4816; A61K 47/38; A61K 47/32; A61K 47/26

(57) ABSTRACT

There is described a prolonged release pharmaceutical dosage form comprising a plurality of coated beads, each of the coated beads comprising: (a) a granule; (b) a first layer coated on the granule, the first layer comprising: (i) hydromorphone or a pharmaceutically acceptable salt thereof, (ii) naloxone or a pharmaceutically acceptable salt thereof, (iii) an antioxidant compound, and (iii) a chelating compound; and (c) a second layer coated on the first layer, the second layer comprising a prolonged release agent. The dosage form has improved stability and dissolution properties. Another aspect of the invention relates to use of a combination of an antioxidant (such as sodium metabisulfite) and a chelating agent (such as ethylenedinitrotetraacetic acid disodium salt dihydrate) to improve the stability and/or dissolution properties of a prolonged release dosage form comprising (i) hydromorphone or a pharmaceutically acceptable salt thereof and (ii) naloxone or a pharmaceutically acceptable salt thereof.

### CROSS-REFERENCE TO RELATED APPLICATION

The present application claims the benefit under 35 U.S.C. §119(e) of provisional patent application Ser. No. 61/796,390, filed Nov. 9, 2012, the contents of which are hereby incorporated by reference.

### BACKGROUND OF THE INVENTION

1. Field of the Invention

In one of its aspect, the present invention relates to a prolonged release pharmaceutical dosage form comprising hydromorphone or a pharmaceutically acceptable salt thereof and naloxone or a pharmaceutically acceptable salt thereof. In another of its aspect, the present invention relates to the use of such a prolonged release pharmaceutical dosage form for use in treating human beings.

2. Description of the Prior Art

Prolonged release pharmaceutical dosage forms represent an important tool in a medical practioner's armoury for treating diseases. One of the general benefits generally attributed to prolonged release pharmaceutical dosage forms versus immediate release pharmaceutical dosage forms includes increased patient compliance as a consequence of reduced administration frequency.

There are various technologies available for obtaining prolonged release dosage forms. Prolonged release properties may be conveyed by so-called prolonged release matrix systems, prolonged release coatings, osmotic dosage forms, multi-layered dosage forms etc.

When developing a prolonged release formulation, it is generally necessary to choose the respective formulation technology with respect to the physico-chemical and physiological properties of the pharmaceutically active agent(s) in question. This means a substantial amount of work for the formulation specialist. This will be even more so where the dosage form comprises pharmaceutically active agents such opioid agonists which theoretically can be abused—i.e., are not used for medicinal purposes.

There is thus a continuing interest in pharmaceutical dosage forms which comprise opioid analgesic as pharmaceutically active agents, which provide prolonged release properties and account for opioids' potential of being abused.

International Publication Number WO 2011/141488 [Danagher et al. (Danagher)] teaches pharmaceutical compositions comprising hydromorphone and naloxone. While the formulation disclosed in Danagher represent an important advance in the art, there is still room for improvement. Specifically, there is room for improvement in one or both of the stability and dissolution properties of some of the specific embodiments of the pharmaceutical compositions exemplified in Danagher.

### SUMMARY OF THE INVENTION

It is an object of the present invention to provide a novel prolonged release pharmaceutical dosage form.

Accordingly, in one of its aspects, the present invention provides a prolonged release pharmaceutical dosage form comprising a plurality of coated beads, each of the coated beads comprising:

(a) a granule;

(b) a first layer coated on the granule, the first layer comprising: (i) hydromorphone or a pharmaceutically acceptable salt thereof, (ii) naloxone or a pharmaceutically acceptable salt thereof, (iii) an antioxidant compound, and (iii) a chelating compound; and

(c) a second layer coated on the first layer, the second layer comprising a prolonged release agent.

In another of its aspects, the present invention provides a coated bead comprising:

(a) a granule;

(b) a first layer coated on the granule, the first layer comprising: (i) hydromorphone or a pharmaceutically acceptable salt thereof, (ii) naloxone or a pharmaceutically acceptable salt thereof, (iii) an antioxidant compound, and (iii) a chelating compound; and

(c) a second layer coated on the first layer, the second layer comprising a prolonged release agent.

In another of its aspects, the present invention provides a prolonged release pharmaceutical dosage form comprising a plurality of coated beads disposed in a hydroxypropyl methyl cellulose capsule, each of the coated beads comprising:

(a) a granule;

(b) a first layer coated on the granule, the first layer comprising: (i) hydromorphone hydrochloride, (ii) naloxone hydrochloride, (iii) an antioxidant compound, and (iii) a chelating compound, wherein (i) and (ii) are present in a weight ratio of about 2:1;

(c) a second layer coated on the first layer, the second layer comprising ethyl cellulose; and

(d) a third layer coated on the second layer, the third layer comprising a polyvinyl alcohol-polyethylene glycol graft copolymer.

In yet another of its aspects, the present invention relates to the use of the combination of an antioxidant (such as sodium metabisulfite) and a chelating agent (such as ethylenedinitrotetraacetic acid disodium salt dihydrate) to improve the stability and/or dissolution properties of a prolonged release dosage form comprising (i) hydromorphone or a pharmaceutically acceptable salt thereof and (ii) naloxone or a pharmaceutically acceptable salt thereof.

The present inventor has discovered that the use of a combination of an antioxidant (such as sodium metabisulfite) and a chelating agent (such as ethylenedinitrotetraacetic acid disodium salt dihydrate) can be used to improved stability and/or dissolution properties (or dissolution profile—these terms are used interchangeably throughout this specification) of a prolonged release dosage form comprising (i) hydromorphone or a pharmaceutically acceptable salt thereof and (ii) naloxone or a pharmaceutically acceptable salt thereof. While the embodiments exemplified below are focussed on such a prolonged release dosage form in the form of coated beads, it is believed that the improvement in stability and/or dissolution properties will also be seen in other dosage forms such as those described in Danagher. Thus, it is believed that that the improvement in stability and/or dissolution properties will also be seen in other dosage forms such as matrix dosage forms and the like comprising (i) hydromorphone or a pharmaceutically acceptable salt thereof and (ii) naloxone or a pharmaceutically acceptable salt thereof as the active ingredients. A non-limiting example of improving the stability of the dosage form includes improving the 24 month shelf life stability of the dosage form.

Antioxidant compound is not particularly restricted.

Preferably, the antioxidant compound is selected from the group consisting of Na metabisulfite, butylated hydroxytoluene (BHT), butylated hydroxyanisole (BHA), propyl gallate (PG), cysteine (CYS), alpha tocopherol, ascorbic acid, phosphoric acid, potassium metabisulfite, sodium ascorbate to provide ascorbic acid, sodium bisulfite, sodium sulfite and any mixture of two or more of these.

Preferably, the antioxidant compound is present in an amount in the range of from about 0.001% to 1.0%, more preferably from about 0.01% to about 1.0%, more preferably from about 0.01% to about 0.1%, more preferably from about 0.01%-0.005%.

In a more preferred embodiment, the antioxidant compound is selected from the group consisting of (preferred amounts in parenthesis) sodium metabisulfite (from about 0.001% to about 1.0%), butylated hydroxytoluene (BHT) (from about 0.01% to about 1.0%), butylated hydroxyanisole (BHA) (from about 0.001% to about 1.0%), propyl gallate (PG) (from about 0.001%-0.1%), cysteine (CYS), alpha tocopherol (from about 0.001% to about 0.05%), ascorbic acid (from about 0.01% to about 0.1%), phosphoric acid (from about 0.001% to about 0.005%), potassium metabisulfite (from about 0.001% to about 1.0%), sodium ascorbate to provide ascorbic acid (from about 0.01% to about 0.1%), sodium bisulfite (from about 0.001% to about 1.0%), sodium sulfite (from about 0.001% to about 1.0%) and any mixture of two or more of these.

The most preferred antioxidant is sodium metabisulfite (preferably used in an amount of from about 0.001% to about 1.0%)

The chelating agent is not particularly restricted.

Preferably the chelating agent is ethylenediaminetetraacetic acid and/or an ethylenediaminetetraacetic acid salt (e.g., EDTA HCl), fumaric acid and any mixture of two or more of these.

When the chelating agent is ethylenediaminetetraacetic acid or an ethylenediaminetetraacetic acid salt (e.g., EDTA HCl), it is preferred that it be used in an amount of from about 0.005% to about 0.1%.

When the chelating agent is fumaric acid, it is preferred that it be used in an amount up to about 0.004%.

The amounts of antioxidant compound and chelating agent expressed above are referred to as %. This is intended to mean weight % of the drug containing portion of the prolonged release dosage form. In the case of a coated beaded embodiment of the present prolonged release dosage form, the active ingredients are typically used in a drug layer and the antioxidant compound/chelating agent amounts described above may be weight % of this drug layer. In the case of a matrix embodiment of the present prolonged release dosage form, the active ingredients are typically mixed with one or more matrix-forming materials to form a matrix composition the antioxidant compound/chelating agent amounts described above may be weight % of this matrix composition.

### DETAILED DESCRIPTION OF THE PREFERRED EMBODIMENTS

The present invention as illustratively described in the following may suitably be practiced in the absence of any element or elements, limitation or limitations, not specifically disclosed herein.

The present invention will be described with respect to particular embodiments and with reference to certain figures but the invention is not limited thereto but only by the claims. Terms as set forth hereinafter are generally to be understood in their common sense unless indicated otherwise.

Where the term “comprising” is used in the present description and claims, it does not exclude other elements. For the purposes of the present invention, the term “consisting of” is considered to be a preferred embodiment of the term “comprising of”. If hereinafter a group is defined to comprise at least a certain number of embodiments, this is also to be understood to disclose a group which preferably consists only of these embodiments.

Where an indefinite or definite article is used when referring to a singular noun, for example, “a”, “an” or “the”, this includes a plural of that noun unless something else is specifically stated.

In the context of the present invention the terms “about” or “approximately” denote an interval of accuracy that the person skilled in the art will understand to still ensure the technical effect of the feature in question. The term typically indicates deviation from the indicated numerical value of ±10%, and preferably of ±5%.

The term “in vitro release” and its grammatical variations as well as similar expression refers to the release rate by which a pharmaceutically active agent, for example, hydromorphone HCl is released from the pharmaceutical composition when the in vitro release rate is tested by the paddle method according to the European Pharmacopeia as described in the Ph. Eur. 2.9.3 6^{th} edition. The paddle speed is typically set at 75 or 100 rpm in 500 ml or 900 ml simulated gastric fluid (SGF) dissolution medium with pH 1.2. Aliquots of the dissolution media are withdrawn at the respective time points and analysed by HPLC with a C18 column, eluted with 30 mM phosphate buffer in acetonitrile (70:70; pH 2.9) with a flow rate of 1.0 ml/min and detected at 220 nm. It is specifically indicated if in the context of the present invention in vitro release rates are determined using a different test method (such as SGF with 40% (v/v) of ethanol).

The amount of dissolution liquid and the rotational speed of the paddle apparatus may depend on the amount of active agent tested. For example, pharmaceutical compositions comprising up to 16 mg hydromorphone HCl may be tested at 75 rpm in 500 ml dissolution liquid while higher dosage strengths may be tested at 100 rpm in 900 ml dissolution liquid.

The term “Simulated Gastric Fluid, pH 1.2” refers to 0.1 N HCl, pH 1.2.

In the context of the present invention, the terms “immediate release” or “conventional release” refer to pharmaceutical compositions showing a release of the active substance(s) which is not deliberately modified by a special formulation design and/or manufacturing methods. For oral dosage forms this means that the dissolution profile of the active substance(s) depends essentially on its (theirs) intrinsic properties. Typically, the terms “immediate release” or “conventional release” refer to pharmaceutical compositions which release in vitro >75% (by weight) of the pharmaceutically active agent(s) at 45 min.

In the context of the present, the terms “prolonged release” and “controlled release” are used interchangeably and refer to pharmaceutical compositions showing a slower release of the active agent(s) than that of a conventional release pharmaceutical composition administered by the same route. Prolonged or controlled release is achieved by a special formulation design and/or manufacturing method. Typically, the terms “prolonged release” and “controlled release refer to pharmaceutical compositions which release in vitro ≦75% (by weight) of the pharmaceutically active agent at 45 min.

Prolonged release properties may be obtained by different means such as by a coating which is then designated as a prolonged release coating.

In order to obtain “prolonged or controlled release” properties, one typically uses materials which are known to prolong the release from a dosage form comprising such as a prolonged release coating. Typical examples of such “prolonged or controlled release materials” are hydrophobic polymers such as ethyl cellulose, hydrophilic polymers such as hydroxypropyl cellulose and the like. The nature of the “prolonged or controlled release material” may depend on whether the release properties are attained by a “prolonged release coating”. The term “prolonged release coating material” indicate that a material is used for obtaining a prolonged release coating.

The terms “prolonged release coating formulation” or “controlled release coating formulation” refer to a pharmaceutical composition including at least one prolonged release material or controlled release material, and at least one hydromorphone and naloxone or the pharmaceutically acceptable salts or derivatives thereof. The terms “prolonged release material” and “controlled release material” can be used interchangeably. In a “prolonged release coating formulation” or “controlled release coating formulation”, the “prolonged release material” or “controlled release material” are disposed on the pharmaceutically active agents to form a diffusion barrier. Typically, unlike in a matrix formulation, the actives are not intimately mixed with the prolonged release material and the prolonged release coating does not form a three dimensional structure within which the actives are distributed. As the term implies, the prolonged release material forms a layer above the actives. The pharmaceutically active agent is released from a prolonged release coating formulation over prolonged periods of time, such as, for example, 8, 10, 12, 14, 16, 18, 20, 22 or 24 hours.

It is to be understood that a material will be considered to act as prolonged or controlled release material if the dissolution profile of the pharmaceutically active agent(s) is slowed down compared to an immediate or conventional release formulation. If a prolonged or controlled release material can be used for manufacturing a prolonged or controlled release coating, it will be considered as a prolonged or controlled release coating material.

Pharmaceutically acceptable excipients which are used to adjust an already prolonged or controlled release to a specific profile are not necessarily considered to be prolonged or controlled release materials.

When it is mentioned that a prolonged release coating is disposed on pharmaceutically active agents, this is not to be construed as meaning that such a coating will necessarily be directly layered on such active pharmaceutically agents. Of course, if pharmaceutically active agents are layered on a carriers such as nu-pareil beads, the coating may be disposed directly thereon.

A pharmaceutical composition with a controlled or prolonged release coating may be obtained by combining the pharmaceutically active agents with carriers such as non-pareil beads and disposing a prolonged release coating on such combinations. Such coating may be made from polymers such cellulose ethers with ethyl cellulose being preferred, acrylic resins, other polymers and mixtures thereof. Such controlled or prolonged release coatings may comprise additional excipients such as pore-formers, binders and the like.

The present invention as disclosed herein with respect to all aspects and embodiments is meant to encompass the use of any pharmaceutically acceptable salt or derivative of hydromorphone and naloxone. Any embodiment of the invention referring to hydromorphone and naloxone is also meant to refer to salts and preferably the hydrochloride salts thereof unless indicated otherwise.

Pharmaceutically acceptable salts include, but are not limited to, inorganic acid salts such as hydrochloride, hydrobromide, sulfate, phosphate and the like; organic acid salts such as formate, acetate, trifluoroacetate, maleate, tartrate and the like; sulfonates such as methanesulfonate, benzenesulfonate, p-toluenesulfonate, and the like; amino acid salts such as arginate, asparginate, glutamate and the like, and metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like.

Pharmaceutically acceptable derivatives of hydromorphone and naloxone include esters thereof as well as modified forms such as glycosylated, pegylated or hesylated forms of hydromorphone and naloxone.

If in the following reference is made to a pharmaceutically active agent such as hydromorphone, this always also includes the reference to a pharmaceutically acceptable salt or derivative of the free base of this pharmaceutically active agent unless it is specifically indicated that the reference to the pharmaceutically active agent, such as use of the term “hydromorphone” should only refer to the free base.

The use of the hydrochloride salts of both hydromorphone and naloxone is preferred.

In a preferred embodiment, the pharmaceutical dosage forms comprise hydromorphone or a pharmaceutically acceptable salt or derivative thereof or naloxone or a pharmaceutically acceptable salt or derivative thereof as the sole pharmaceutically active agents.

The pharmaceutical compositions may comprise about 1 to about 64 mg such as about 1 mg, about 2 mg, 3 mg, about 4 mg, about 8 mg, about 12 mg, about 16 mg, about 24 mg, about 32 mg, about 40 mg, about 48 mg or about 64 mg hydromorphone hydrochloride or equimolar amounts of any other pharmaceutically acceptable salt or derivative including but not limited to hydrates and solvates or of the free base. Where reference is made to amounts of hydromorphone hydrochloride this relates to anhydrous hydromorphone hydrochloride. If a hydrated version of hydromorphone hydrochloride is used, this will be used in an amount equivalent to the afore-mentioned amounts of anhydrous hydromorphone hydrochloride.

The pharmaceutical compositions may comprise about 0.5 to about 256 mg, such as about 0.5 mg, about 0.75 mg, about 1 mg, about 1.5 mg, about 2 mg, about 4 mg, about 8 mg, about 12 mg, about 16 mg, about 24 mg, about 32 mg, about 48 mg, about 64 mg, about 96 mg, about 128 or about 256 mg of naloxone hydrochloride or equimolar amounts of any other pharmaceutically acceptable salt, derivative or form including but not limited to hydrates and solvates or of the free base. Where reference is made to amounts of naloxone hydrochloride this relates to anhydrous naloxone hydrochloride. If a hydrated version of naloxone hydrochloride is used, this will be used in an amount equivalent to the afore-mentioned amounts of anhydrous naloxone hydrochloride.

In some embodiments, the present invention is directed to a prolonged release pharmaceutical coated bead composition comprising at least hydromorphone or a pharmaceutically acceptable salt or derivative thereof or naloxone or a pharmaceutically acceptable salt or derivative thereof and at least one prolonged release material which is preferably combined with these pharmaceutically active agents; wherein the amount of hydromorphone or a pharmaceutically acceptable salt or derivative thereof and/or naloxone or a pharmaceutically acceptable salt or derivative thereof released in vitro in 500 or 900 ml of Simulated Gastric Fluid, pH 1.2 using the Ph. Eur. paddle method at 100 rpm at 37° C. is:

at 1 h: 25 to 55% by weight of the pharmaceutically active agents,

at 2 h: 45 to 75% by weight of the pharmaceutically active agents,

at 3 h: 55 to 85% by weight of the pharmaceutically active agents,

at 4 h: 60 to 90% by weight of the pharmaceutically active agents,

at 6 h: 70 to 100% by weight of the pharmaceutically active agents,

at 8 h: more than 85% by weight of the pharmaceutically active agents,

at 10 h: more than 90% by weight of the pharmaceutically active agents.

The pharmaceutically active agents may preferably be hydromorphone HCl and naloxone HCl being preferred. The prolonged release pharmaceutical composition may comprise these actives in the above indicated amounts and in a weight ratio range of from about 2:1 to about 1:2 such as a weight ratio of about 2:1, about 1:1 or about 1:2.

In some embodiments, the present invention is directed to a prolonged release pharmaceutical coated bead composition comprising at least hydromorphone or a pharmaceutically acceptable salt or derivative thereof or naloxone or a pharmaceutically acceptable salt or derivative thereof and at least one prolonged release material; wherein the amount of hydromorphone and/or a pharmaceutically acceptable salt or derivative thereof or naloxone or a pharmaceutically acceptable salt or derivative thereof released in vitro in 500 or 900 ml of Simulated Gastric Fluid, pH 1.2 using the Ph. Eur. paddle method at 100 rpm at 37° C. is:

at 1 h: 30 to 50% by weight of the pharmaceutically active agents,

at 2 h: 50 to 70% by weight of the pharmaceutically active agents,

at 3 h: 60 to 80% by weight of the pharmaceutically active agents,

at 4 h: 65 to 85% by weight of the pharmaceutically active agents,

at 6 h: 75 to 95% by weight of the pharmaceutically active agents,

at 8 h: more than 90% by weight of the pharmaceutically active agents,

at 10 h: more than 95% by weight of the pharmaceutically active agents.

The pharmaceutically active agents may preferably be hydromorphone HCl and naloxone HCl being preferred. The prolonged release pharmaceutical composition may comprise these actives in the above indicated amounts and in a weight ratio range of from about 2:1 to about 1:2 such as a weight ratio of about 2:1, about 1:1 or about 1:2.

In some embodiments, the present invention is directed to a prolonged release pharmaceutical coated bead composition comprising at least hydromorphone or a pharmaceutically acceptable salt or derivative thereof or naloxone or a pharmaceutically acceptable salt or derivative thereof and at least one prolonged release material which is preferably combined with these pharmaceutically active agents; wherein the amount of hydromorphone or a pharmaceutically acceptable salt or derivative thereof and/or naloxone or a pharmaceutically acceptable salt or derivative thereof released in vitro in 500 or 900 ml of Simulated Gastric Fluid, pH 1.2 using the Ph. Eur. paddle method at 100 rpm at 37° C. is:

at 1 h: 10 to 30% by weight of the pharmaceutically active agents,

at 2 h: 34 to 54% by weight of the pharmaceutically active agents,

at 3 h: 53 to 73% by weight of the pharmaceutically active agents,

at 4 h: 65 to 85% by weight of the pharmaceutically active agents,

at 6 h: 75 to 95% by weight of the pharmaceutically active agents,

at 8 h: 80 to 100% by weight of the pharmaceutically active agents,

at 10 h: more than 90% by weight of the pharmaceutically active agents.

The pharmaceutically active agents may preferably be hydromorphone HCl and naloxone HCl being preferred. The prolonged release pharmaceutical composition may comprise these actives in the above indicated amounts and in a weight ratio range of from about 2:1 to about 1:2 such as a weight ratio of about 2:1, about 1:1 or about 1:2.

In some embodiments, the present invention is directed to a prolonged release pharmaceutical coated bead composition comprising at least hydromorphone or a pharmaceutically acceptable salt or derivative thereof or naloxone or a pharmaceutically acceptable salt or derivative thereof and at least one prolonged release material which is preferably combined with these pharmaceutically active agents; wherein the amount of hydromorphone or a pharmaceutically acceptable salt or derivative thereof and/or naloxone or a pharmaceutically acceptable salt or derivative thereof released in vitro in 500 or 900 ml of Simulated Gastric Fluid, pH 1.2 using the Ph. Eur. paddle method at 100 rpm at 37° C. is:

at 1 h: 5 to 45% by weight of the pharmaceutically active agents,

at 2 h: 15 to 55% by weight of the pharmaceutically active agents,

at 3 h: 30 to 70% by weight of the pharmaceutically active agents,

at 4 h: 35 to 75% by weight of the pharmaceutically active agents,

at 6 h: 40 to 80% by weight of the pharmaceutically active agents,

at 8 h: 50 to 90% by weight of the pharmaceutically active agents,

at 10 h: 60 to 100% by weight of the pharmaceutically active agents,

at 12 h: 65 to 100% by weight of the pharmaceutically active agents.

The pharmaceutically active agents may preferably be hydromorphone HCl and naloxone HCl being preferred. The prolonged release pharmaceutical composition may comprise these actives in the above indicated amounts and in a weight ratio range of from about 2:1 to about 1:2 such as a weight ratio of about 2:1, about 1:1 or about 1:2.

Preferably, the amount of the pharmaceutically active agents released in vitro in 500 or 900 ml of Simulated Gastric Fluid, pH 1.2 using the Ph. Eur. paddle method at 100 rpm at 37° C. is:

at 1 h: 8 to 42% by weight of the pharmaceutically active agents,

at 2 h: 18 to 52% by weight of the pharmaceutically active agents,

at 3 h: 33 to 67% by weight of the pharmaceutically active agents,

at 4 h: 38 to 72% by weight of the pharmaceutically active agents,

at 6 h: 43 to 77% by weight of the pharmaceutically active agents,

at 8 h: 53 to 87% by weight of the pharmaceutically active agents,

at 10 h: 63 to 97% by weight of the pharmaceutically active agents,

at 12 h: 73 to 100% by weight of the pharmaceutically active agents.

The pharmaceutically active agents may preferably be hydromorphone HCl and naloxone HCl being preferred. The prolonged release pharmaceutical composition may comprise these actives in the above indicated amounts and in a weight ratio range of from about 2:1 to about 1:2 such as a weight ratio of about 2:1, about 1:1 or about 1:2.

More preferably, the amount of the pharmaceutically active agents released in vitro in 500 or 900 ml of Simulated Gastric Fluid, pH 1.2 using the Ph. Eur. paddle method at 100 rpm at 37° C. is:

at 1 h: 15 to 37% by weight of the pharmaceutically active agents,

at 2 h: 25 to 47% by weight of the pharmaceutically active agents,

at 3 h: 38 to 62% by weight of the pharmaceutically active agents,

at 4 h: 42 to 66% by weight of the pharmaceutically active agents,

at 6 h: 50 to 74% by weight of the pharmaceutically active agents,

at 8 h: 60 to 84% by weight of the pharmaceutically active agents,

at 10 h: 68 to 92% by weight of the pharmaceutically active agents,

at 12 h: 78 to 100% by weight of the pharmaceutically active agents.

The pharmaceutically active agents may preferably be hydromorphone HCl and naloxone HCl being preferred. The prolonged release pharmaceutical composition may comprise these actives in the above indicated amounts and in a weight ratio range of from about 2:1 to about 1:2 such as a weight ratio of about 2:1, about 1:1 or about 1:2.

Even more preferably, the amount of the pharmaceutically active agents released in vitro in 500 or 900 ml of Simulated Gastric Fluid, pH 1.2 using the Ph. Eur. paddle method at 100 rpm at 37° C. is:

at 1 h: 19 to 33% by weight of the pharmaceutically active agents,

at 2 h: 29 to 43% by weight of the pharmaceutically active agents,

at 3 h: 43 to 47% by weight of the pharmaceutically active agents,

at 4 h: 47 to 61% by weight of the pharmaceutically active agents,

at 6 h: 55 to 69% by weight of the pharmaceutically active agents,

at 8 h: 65 to 79% by weight of the pharmaceutically active agents,

at 10 h: 73 to 87% by weight of the pharmaceutically active agents,

at 12 h: 83 to 100% by weight of the pharmaceutically active agents.

The pharmaceutically active agents may preferably be hydromorphone HCl and naloxone HCl being preferred. The prolonged release pharmaceutical composition may comprise these actives in the above indicated amounts and in a weight ratio range of from about 2:1 to about 1:2 such as a weight ratio of about 2:1, about 1:1 or about 1:2.

Even more preferably, the amount of the pharmaceutically active agents released in vitro in 500 or 900 ml of Simulated Gastric Fluid, pH 1.2 using the Ph. Eur. paddle method at 100 rpm at 37° C. is:

at 1 h: 1 to 15% by weight of the pharmaceutically active agents,

at 2 h: 6 to 26% by weight of the pharmaceutically active agents,

at 3 h: 15 to 35% by weight of the pharmaceutically active agents,

at 4 h: 25 to 45% by weight of the pharmaceutically active agents,

at 6 h: 40 to 60% by weight of the pharmaceutically active agents,

at 8 h: 55 to 75% by weight of the pharmaceutically active agents,

at 10 h: 60 to 80% by weight of the pharmaceutically active agents,

at 12 h: 70 to 100% by weight of the pharmaceutically active agents.

The pharmaceutically active agents may preferably be hydromorphone HCl and naloxone HCl being preferred. The prolonged release pharmaceutical composition may comprise these actives in the above indicated amounts and in a weight ratio range of from about 2:1 to about 1:2 such as a weight ratio of about 2:1, about 1:1 or about 1:2.

Storage under stressed conditions in the context of the present invention means that a pharmaceutical composition is subjected to increased temperature and/or relative humidity (RH) for prolonged periods of time. For example, typical stressed conditions refer to storage over at least one, two, three, four, five, six, twelve or eighteen months at 25° C. and 60% RH. Other stressed conditions refer to storage over at least one, two, three, four, five, six or twelve months at 30° C. and 65% RH Other stressed conditions refer to storage over at least one, two, three, four, five or six months at 40° C. and 75% RH.

Such stressed storage conditions are used to determine whether a pharmaceutical composition has a shelf life sufficient for long time storage under conditions as they are common in patients' households without negative effects on its safety and efficacy. Such negative effects may include that the in-vitro release rates change over time so that the efficacy of the composition is affected as different amounts of actives are released after administration. Similarly, negative effects may also result from degradation of the pharmaceutically active agents which may either decrease the overall amount of functional pharmaceutically active agent or lead to formation of toxic by-products.

If changes in the in vitro release profile or with respect to the amount of the active agent(s) of a pharmaceutical composition are observed after storage under stressed conditions, this may be indicative of stability problems. If such changes are not observed, this means vice versa that the pharmaceutical composition is storage stable.

The above mentioned stressed storage conditions can be used to estimate whether a pharmaceutical dosage will have a shelf life of at least about 12 months, at least about 18 months, at least about 24 months or at least about 36 months. Usually a shelf life of 18 months or more may be desirable as this is usually better compatible with e.g. supply of excipients, actives etc. for manufacturing purposes. If a pharmaceutical composition is storage stable, i.e. has essentially the same release rate after storage over at least one, two, three, four, five or more months at 25° C. and 60% RH, this will be usually indicative of shelf life of at least about 12 months. If a pharmaceutical composition is storage stable, i.e. has essentially the same release rate after storage over at least one, two, three, four, five or more months at 30° C. and 65% RH, this will be usually indicative of shelf life of at least about 18 months. If a pharmaceutical composition is storage stable, i.e. has essentially the same release rate after storage over at least one, two, three, four, five or more months at 40° C. and 75% RH, this will be usually indicative of a shelf life of at least about 24 months such as 36 months.

The term “substantially the same release rate” refers to the situation where the in vitro release rate for a pharmaceutical composition which has been subjected to stressed conditions is compared to a reference composition. The reference composition is an identical pharmaceutical composition which, however, has not been subjected to stressed conditions. If the in vitro release profile of the composition subjected to stressed conditions does not deviate by more than about 20%, preferably by no more than about 15%, more preferably by no more than 10% and even more preferably by no more than about 5% from the in vitro release profile of the reference composition, the in-vitro release rate is considered to be substantially the same.

The term “hydromorphone and/or naloxone related substances” or the like refers to substances that arise from chemical reactions of hydromorphone or naloxone, pharmaceutically acceptable salts and derivatives thereof such as e.g. degradation. These substances can be distinguished as known hydromorphone related substances where the identity of the substance and its origin is known, as known naloxone related substances where the identity of the substance and its origin is known, and as unknown substances. For unknown substances, their identity is not known. However, it is assumed that they arise from hydromorphone and/or naloxone, pharmaceutically acceptable salts and derivatives thereof. It is to be understood that the term “hydromorphone and naloxone related substances” includes the sum of known hydromorphone related substances, known naloxone related substances and unknown substances and is thus equivalent to the term “total hydromorphone and naloxone related substances”.

Terms like “less than about 4% of substances related to hydromorphone and naloxone, or to pharmaceutically acceptable salts or derivatives thereof” or “less than about 3% of substances related to hydromorphone and naloxone or to pharmaceutically acceptable salts or derivatives thereof” etc. indicate that the amount of total substances as described in the preceding paragraph is less than e.g. 4% or 3% by weight based on the total amount of the active ingredient which is present in lower amounts (i.e. hydromorphone or naloxone), or a pharmaceutically acceptable salt or derivative thereof which is present in the pharmaceutical composition in the lower amount. Thus, if a pharmaceutical composition comprises hydromorphone HCl and naloxone HCl in 1:2 ratio by weight, the amount of total substances is calculated from the sum of known hydromorphone HCl related substances, known naloxone HCl related substances and unknown substances which is then referenced to the amount of hydromorphone HCl. If a pharmaceutical composition comprises hydromorphone HCl and naloxone HCl in 2:1 ratio by weight, the amount of total substances is calculated from the sum of known hydromorphone HCl related substances, known naloxone HCl related substances and unknown substances which is then referenced to the amount of naloxone HCl.

“Known hydromorphone related substances” include hydromorphone n-oxide, noroxymorphone, pseudohydromorphone.

“Known naloxone related substances” include noroxymorphon, 10a-hydroxynaloxon, 7,8-didehydronaloxon, pseudonaloxon, 3-o-allylnaloxon.

Terms like “less than 4% of known substances related to hydromorphone, or to pharmaceutically acceptable salts or derivatives thereof” or “less than 3% of known substances related to hydromorphone, or to pharmaceutically acceptable salts or derivatives thereof” etc. indicate that the amount of known hydromorphone related substances is less than e.g. 4% or 3% of known hydromorphone related substance by weight based on the total amount of hydromorphone, or a pharmaceutically acceptable salt or derivative thereof in the composition.

Terms like “less than 4% of known substances related to naloxone, or to pharmaceutically acceptable salts or derivatives thereof” or “less than 3% of known substances related to naloxone, or to pharmaceutically acceptable salts or derivatives thereof” etc. indicate that the amount of known naloxone related substances is less than e.g. 4% or 3.0% of known naloxone related substance by weight based on the total amount of naloxone, or a pharmaceutically acceptable salt or derivative thereof in the composition.

In order to assess stability one may subject a pharmaceutical composition to stressed conditions as mentioned above and determine the amount of total hydromorphone and/or naloxone related substances. One then determines the amount of total hydromorphone and/or naloxone related substances for an identical pharmaceutical composition which has not been subjected to stressed conditions. This composition is considered to be a reference composition. The detection of “total hydromorphone related and/or naloxone substances” is typically performed by HPLC analysis using e.g. CAT columns. The amount of the substances including the amount of unknown substances is then determined by calculating the area under the respective peaks in the chromatogram. The identity of substances can be determined by doing the same analysis with pure known reference substances. In a further aspect the present invention aims at providing pharmaceutical compositions which after storage under stressed conditions have less than 4%, less than 3%, less than 2%, less than 1%, less than 0.5%, less than 0.2% or even less than 0.1% of total substances related to hydromorphone or a pharmaceutically acceptable salt or derivative thereof and/or related to naloxone or a pharmaceutically acceptable salt or derivative thereof.

In a further aspect the present invention aims at providing pharmaceutical compositions which after storage under stressed conditions have less than 1% such as less than 0.5%, less than 0.4%, less than 0.3%, less than 0.2%, less than 0.1% or even less than 0.05% of known substances related to hydromorphone or a pharmaceutically acceptable salt or derivative thereof and less than 1% such as less than 0.5% of known substances related to naloxone or a pharmaceutically acceptable salt or derivative thereof.

Stressed storage conditions may be the same as mentioned above. Thus typical stressed conditions may refer to storage over at least one, two, three, four, five or six months at 25° C. and 60% RH, at 30° C. and 65% RH or at 40° C. and 75% RH.

A pharmaceutical composition will thus be considered to be stable if after subjecting it to stressed conditions, it has no more than about 4% such as no more than about 3%, preferably no more than about 2%, more preferably no more than about 1% and even more preferably no more than about 0.5% of hydromorphone and/or naloxone related substances.

The prolonged release compositions in accordance with the invention may be formulated into different dosage forms. For example, prolonged release compositions may take the form of tablets or mini-tablets. Tablets may be a monolithic tablet comprising, for example, a continuous prolonged release matrix. However, tablets or mini-tablets may be also be made from multiparticulates which are compressed into tablets. Such multiparticulates may, for example, comprise a prolonged release matrix optionally with an immediate release phase or active loaded beads with a prolonged release coating and optionally an immediate release phase thereon. The dosage form may also take the form of such multiparticulates, for example, granules or mini-tablets which may be filled into a capsule.

The in vitro release rates of the prolonged release pharmaceutical compositions will be chosen such that a therapeutic efficacy in vivo is achieved over preferably at least twelve hours and in some instance even up to twenty four hours. Such compositions may be described as “twice a day” or “once a day” formulations as they may be administered on such a regimen.

The prolonged release material may be any material that is known to be capable of imparting controlled release properties on the active agent.

Such materials may be hydrophilic and/or hydrophobic materials such as gums, cellulose ethers, acrylic polymers, protein-derived materials and the like.

Prolonged materials may also include fatty acids, fatty alcohols, glyceryl esters of fatty acids, polyethylene glycols, mineral and oils and waxes. Fatty acids and fatty alcohols preferable are those with a C₁₀ to C₃₀ chain, preferably with a C₁₂ to C₂₄ chain and more preferably with a C₁₄ to C₂₀ chain or a C₁₆ to C₂₀ chain. Materials such as stearyl alcohol, cetostearyl alcohol, cetyl alcohol, myristyl alcohol and polyalkylene glycols may be preferred. Waxes may be selected from natural and synthetic waxes such as beeswax, carnauba wax. Oils may be vegetable oils and include, for example, castor oil.

The prolonged release matrix materials which may be considered in the context of the present invention may also be selected from cellulose ethers.

The term “cellulose ethers” comprises cellulose-derived polymers derivatized with at least alkyl and/or hydroxyalkyl groups which may be hydrophilic or hydrophobic.

For example, the prolonged release matrix material may be a hydrophilic hydroxy alkyl cellulose such as a hydroxy (C₁-C₆) alkyl celluloses such as hydroxypropyl cellulose, hydroxypropylmethyl cellulose and particularly preferably hydroxyethyl cellulose.

Examples of hydrophobic cellulose ethers include e.g. ethyl cellulose. The use of ethyl cellulose may be preferred. Hydrophobic cellulose ethers such as ethyl cellulose may be particularly suitable for imparting alcohol resistance to pharmaceutical compositions.

A particularly suitable material for prolonged release matrix formulations in accordance with the present invention may be selected from the group of acrylic resins. Such acrylic resins may be made from (meth)acrylic acid (co) polymers.

There are various types of (meth)acrylic acid (co)polymers available which may be characterised according to the nature of their residues such as neutral (meth)acrylic acid (co)polymers, (meth)acrylic acid (co)polymers with anionic residues or (meth)acrylic acid ester copolymers with cationic residues.

Neutral (meth)acrylic acid (co)polymers include polymers having 95 to 100% by weight of polymerised monomers having neutral residues. Monomers with neutral residues can be C₁-C₄ alkyl esters of acrylic or methacrylic acid such as methylmethacrylate, ethylmethacrylate, butylmethacrylate, methylacrylate, ethylacrylate and butylacrylate. For example, neutral (meth)acrylic acid (co)polymers may comprise 20 to 40% by weight ethylacrylate and 60 to 80% by weight methylmethacrylate. Such polymers are, for example, available under the trade name Eudragit® NE which is a copolymer of 30% by weight ethylacrylate and 70% by weight methylmethacrylate. This polymer is usually provided in the form of a 30% or 40% aqueous dispersion (Eudragit® NE 30 D, Eudragit® NE 40 D or Eudragit® NM 30 D).

(Meth)acrylic acid (co)polymers with functional anionic residues may be (meth)acrylic acid (co)polymers having 25 to 95% by weight of radically polymerised C₁ to C₄ alkyl esters of acrylic or methacrylic acid and 5 to 75% by weight of methacrylate monomers with an anionic group in the alkyl residue. C₁ to C₄ alkyl esters of acrylic or methacrylic acid are again methylmethacrylate, ethyl methacrylate, butylmethacrylate, methylacrylate, ethylacrylate and butylacrylate. A (meth)acrylate monomer with an anionic group in the alkyl residue may be for example acrylic acid and preferably methacrylic acid. Such methacrylic acid copolymers with an anionic functional group may comprise e.g. 40 to 60% by weight methacrylic acid and 60 to 40% by weight methylmethacrylate or 60 to 40% by weight ethyl acrylate. These types of polymers are available as Eudragit® L100/Eudragit® L 12.5 or Eudragit® L 100-55/Eudragit® L 30 D-55, respectively.

For example, Eudragit® L 100 is a copolymer of 50% by weight methylmethacrylate and 50% by weight methacrylic acid. It is also provided as a 12.5% solution (Eudragit® L 12.5). Eudragit® L 100-55 is a copolymer of 50% by weight ethylacrylate and 50% by weight methacrylic acid. It is also provided as 30% dispersion (Eudragit® L 30 D-55).

(Meth)acrylic acid (co)polymers with an anionic functional group may also comprise 20 to 40% by weight methacrylic acid and 80 to 60% by weight methylmethacrylate. These types of polymers are usually available under the trade name Eudragit® S. It is also provided as a 12.5% solution (Eudragit® S 12.5). Another type of methacrylic acid copolymers with an anionic functional group is available under the trade name Eudragit® FS which typically comprises 10 to 30% by weight methylmethacrylate, 50 to 70% by weight methylacrylate and 5 to 15% by weight methacrylic acid. Thus, Eudragit® FS may be a polymer of 25% by weight methylmethacrylate, 65% by weight methylacrylate and 10% by weight methacrylic acid. It is usually provided as 30% dispersion (Eudragit® FS 30 D).

(Meth)acrylic acid (co)polymers with functional cationic groups may be methacrylic acid copolymers with tertiary amino groups. Such polymers may comprise 30% to 80% by weight of radically polymerised C₁-C₄ alkyl esters of acrylic acid or methacrylic acid and 70 to 20% by weight methacrylate monomers with a tertiary amino group in the alkyl rest.

Suitable monomers with a functional tertiary amino group are disclosed, for example, in U.S. Pat. No. 4,705,695 (see column 3, line 64 to column 4, line 13). They include for example dimethylaminoethyl acrylate, 2-dimethylaminopropyl acrylate, dimethylaminopropyl methacrylate, dimethylaminobenzyl acrylate, dimethylaminobenzyl methacrylate, (3-dimethylamino-2,2-dimethyl)propyl acrylate, dimethylamino-2,2-dimethylpropylmethacrylate, (3-diethylamino-2,2-dimethyl)propyl acrylate and diethylamino-2,2-dimethylpropylmethacrylate. Particularly suitable is dimethylaminoethyl methacrylate. The amount of monomers with a tertiary amino group in the copolymer may vary between 20 to 70%, between 40 to 60%. The amount of C₁ to C₄ alkyl esters of acrylic or methacrylic acid may be within 70 to 30% by weight. C₁ to C₄ alcohol esters of acrylic or methacrylic acid include methylmethacrylate, ethylmethacrylate, butylmethacrylate, methylacrylate, ethylacrylate and butylacrylate. A common (meth)acrylic acid (co)polymer with a tertiary amino group may comprise 20 to 30% by weight methylmethacrylate, 20 to 30% by weight butylmethacrylate and 60 to 40% by weight dimethylaminoethyl methacrylate. For example the commercially available Eudragit® E 100 comprises 25% by weight methylmethacrylate, 25% by weight butylmethacrylate and 50% by weight dimethylaminoethyl methacrylate. Another common commercially available polymer, Eudragit® E PO comprises copolymers of methylmethacrylate, butylmethacrylate and dimethylaminoethyl methacrylate in a ratio of 25:25:50.

Another type of (meth)acrylic acid (co)polymers with functional cationic groups is (meth)acrylic acid (co)polymers with a quaternary amino group. This type of (meth)acrylic acid (co)polymers typically comprises 50 to 70% of radically polymerised methylmethacrylate, 20 to 40% by weight of ethylacrylate and 12 to 2% by weight of 2-trimethylammoniumethyl methacrylate chloride. Such polymers are, for example, available under the trade names Eudragit® RS or Eudragit® CRL.

For example, Eudragit® RS comprises radically polymerised units of 65% by weight methylmethacrylate, 30% by weight ethylacrylate and 5% by weight 2-trimethylamoniumethyl methacrylate chloride. Eudragit® RL comprises radically polymerised units of 60% by weight methylmethacrylate, 30% by weight ethylacrylate and 10% by weight 2-trimethylamoniumethyl methacrylate chloride.

The amount of prolonged release material(s) in the prolonged release formulation may be of about 5 to 90% by weight, of about 10 to 70% by weight, of about 20 to 60% by weight, of about 20% to about 55% by weight, of about 25% to about 50% by weight, of about 25% to about 45% by weight and preferably of about 30 to about 40% by weight based on the weight of the pharmaceutical composition. The amount of prolonged release material that is incorporated into the composition can be one way of adjusting the prolonged release properties. For example, if the amount of prolonged release material is increased, the release can be further prolonged. The aforementioned amounts refer to the overall content of prolonged release materials in a pharmaceutical composition. These amounts may thus refer to a mixture of various prolonged release materials such as a neutral (meth)acrylic acid (co)polymer, a hydrophobic cellulose ether and/or a fatty alcohol.

If cellulose ether is among the prolonged release materials, it will typically be present in an amount of about 5% to about 50% by weight, of about 5% to about 45% by weight, of about 5% to about 40% by weight, of about 5% to about 35% by weight, of about 5% to about 30% by weight, of about 5% to about 25% by weight, of about 5% to about 20% by weight such as of about 5% by weight, of about 7% by weight, of about 10% by weight, of about 15% by weight, of about 18% by weight or of about 20% by weight based on the weight of the pharmaceutical composition.

If fatty alcohol is among the prolonged release materials, it will typically be present in an amount of about 5% to about 50% by weight, of about 5% to about 45% by weight, of about 5% to about 40% by weight, of about 5% to about 35% by weight, of about 10% to about 30% by weight, of about 10% to about 25% by weight such as of about 10% by weight, of about 15% by weight, of about 20% by weight or about 25% by weight based on the weight of the pharmaceutical composition.

If (meth)acrylic acid (co)polymer is among the prolonged release materials, it will typically be present in an amount of about 5% to about 50% by weight, of about 5% to about 45% by weight, of about 5% to about 40% by weight, of about 5% to about 35% by weight, of about 10% to about 30% by weight, of about 10% to about 25% by weight such as of about 10% by weight, of about 15% by weight, of about 20% by weight or about 25% by weight based on the weight of the pharmaceutical composition.

The pharmaceutical compositions in accordance with the invention may also include pharmaceutically acceptable excipients such fillers, lubricants, binders, release rate modifiers, agents etc.

Fillers which may also be designated as diluents may include e.g. lactose, preferably anhydrous lactose, glucose or saccharose, starches, their hydrolysates, microcrystalline cellulose, cellatose, sugar alcohols such as sorbitol or mannitol, polysoluble calcium salts like calcium hydrogen phosphate, dicalcium- or tricalcium phosphate and combinations of two or more of the above fillers.

It has been observed that the combination of hydromorphone and naloxone can be moisture sensitive in particular if cellulose ethers are used as prolonged release material. In view of this situation it can be preferred to use fillers which do not import moisture e.g. in the form of water. In preferred embodiments one may thus use anhydrous fillers such as anhydrous lactose.

Lubricants can include highly dispersed silica, talcum, corn starch, magnesium oxide and magnesium- or calcium stearate, fats like hydrated castor oil, sodium stearyl fumarate and combinations of two or more of the above lubricants.

It can be preferred to use a combination of magnesium stearate and talcum as lubricants. It has been found that if appropriate amounts of these lubricants are chosen, one can e.g. improve flow properties of granules used for compressing.

It thus can be preferred to use a lubricant amount of about 0.5% to about 4% by weight, of about 0.7% to about 3% by weight, of about 1% to about 2% by weight such as of about 1.0% by weight, of about 1.1% by weight, of about 1.2% by weight, of about 1.3% by weight, of about 1.4% by weight, of about 1.5% by weight, of about 1.6% by weight, of about 1.7% by weight, of about 1.8% by weight, of about 1.9% by weight or of about 2.0% by weight based on the weight of the pharmaceutical composition. An amount of about 0.75% to about 1.25% by weight based on the weight of the pharmaceutical composition can be preferred, particularly if magnesium stearate and talc are used. The aforementioned amounts refer to the amount of all lubricants (i.e., including mixtures) in the composition.

Binders can include hydroxypropyl cellulose (HPC), hydroxypropyl methyl cellulose, polyvinyl pyrollidone, carbopol, and combinations thereof.

It can be preferred to use HPC as a binder as this may positively influence the hardness of the tablets.

It thus can be preferred to use a binder amount of about 1% to about 10% by weight, of about 2% to about 9% by weight, of about 3% to about 7% by weight, of about 3% to about 6% by weight, of about 4% to about 5% by weight such as of about 4.0% by weight, of about 4.1% by weight, of about 4.2% by weight, of about 4.3% by weight, of about 4.4% by weight, of about 4.5% by weight, of about 4.6% by weight, of about 4.7% by weight, of about 4.8% by weight, of about 4.9% by weight or of about 5.0% by weight based on the weight of the pharmaceutical composition. An amount of about 4.4% to about 5.0% by weight based on the weight of the pharmaceutical composition can be preferred, particularly of HPC is used as binder. The aforementioned amounts refer to the amount of all binders (i.e. including mixtures) in the composition.

It can be preferred to not use povidone as a binder.

Release rate modifiers are pharmaceutically acceptable excipients which may be used to tune the release which otherwise would be obtained using the prolonged release materials, e.g. to accelerate the release or to further slow it down. Such release modifiers may be hydrophilic substances such as polyethylenglycols, hydroxypropylmethlycellulose, hydroxyethylcellulose, and the like or hydrophobic substances such as oils, waxes and the like. Other release modifiers may include some the aforementioned (meth)acrylic acid(co)polymers such as polymers of the Eudragit® RLPO type or gums such as xanthan gum.

Release rate modifiers such as polymers of the Eudragit/® RLPO type, low molecular weight hydroxypropylmethlycellulose such Hypromellose K100M or xanthan gum may be preferred.

Such release rate modifiers may be present in an amount of about 1% to about 20% by weight, of about 2% to about 19% by weight, of about 3% to about 18% by weight, of about 4% to about 17% by weight, of about 5% to about 15% by weight such as of about 5% by weight, of about 6% by weight, of about 7% by weight, of about 8% by weight, of about 9% by weight, of about 10% by weight, of about 11% by weight, of about 12% by weight, of about 13% by weight, of about 14% by weight or of about 15% by weight based on the weight of the pharmaceutical composition. The aforementioned amounts refer to the amount of all release rate modifiers (i.e. including mixtures) in the composition.

It is to be understood that the functions of pharmaceutically acceptable excipients may be overlapping. For example, a spheronising agent such as microcrystalline cellulose can also be used as filler if appropriate amounts are chosen. Further, HPMC may not only act as release rate modifying agent but also as binder if e.g. used in prolonged release formulation with a coating.

Prolonged release coatings may be made from materials which are common in the art.

They may thus be selected from e.g. prolonged release materials selected e.g. from (i) an alkylcellulose; (ii) an acrylic polymer; (iii) polyvinylalcohol or (iv) mixtures thereof. Hydrophobic representatives of the afore-mentioned groups can be preferred. The coating may be applied in the form of an organic or aqueous solution or dispersion.

In some embodiments, the controlled release coating is derived from an aqueous dispersion of the hydrophobic controlled release material. The coated composition can then be cured.

In preferred embodiments, the controlled release coatings include a plasticizer such as those described herein below.

In certain embodiments, one may coat with an amount of coating material which is sufficient to obtain a weight gain level from about 2 to about 20%, e.g., about 2 to about 15% and preferably about 5 to about 10% such as 6%, 7%, 8% or 9% in order to obtain sufficiently prolong the release from the formulation.

Cellulosic materials and polymers, including alkyl celluloses are prolonged release materials well suited for coating substrates, e.g., beads, granules, tablets, etc. according to the invention. Simply by way of example, one preferred alkyl cellulosic polymer is ethyl cellulose.

One commercially available aqueous dispersion of ethyl cellulose is Aquacoat® such as Aquacoat® ECD30 (FMC Corp., Philadelphia, Pa., U.S.A.). Aquacoat is prepared by dissolving the ethyl cellulose in a water-immiscible organic solvent and then emulsifying the same in water in the presence of a surfactant and a stabilizer. After homogenization to generate submicron droplets, the organic solvent is evaporated under vacuum to form a pseudo latex.

Another aqueous dispersion of ethyl cellulose is commercially available as Surelease® (Colorcon, Inc., West Point, Pa., U.S.A.). This product is prepared by incorporating plasticizer into the dispersion during the manufacturing process. A hot melt of a polymer, plasticizer (dibutyl sebacate or medium chain triglycerides), and stabilizer (oleic acid) is prepared as a homogeneous mixture, which is then diluted with an alkaline solution to obtain an aqueous dispersion which can be applied directly onto substrates.

In other of the present invention, the prolonged release coating material is a pharmaceutically acceptable acrylic polymer, including but not limited to acrylic acid and methacrylic acid copolymers, methyl methacrylate copolymers, ethoxyethyl methacrylates, cynaoethyl methacrylate, poly(acrylic acid), poly(methacrylic acid), methacrylic acid alkylamide copolymer, poly(methyl methacrylate), polymethacrylate, poly(methyl methacrylate) copolymer, polyacrylamide, aminoalkyl methacrylate copolymer, poly(methacrylic acid anhydride) and glycidyl methacrylate copolymers.

In certain preferred embodiments, the acrylic polymer is comprised of one or more ammonium methacrylate copolymers. Ammonium methacrylate copolymers are well known in the art, and are described as fully polymerized copolymers of acrylic and methacrylic acid esters with a low content of quaternary ammonium groups. Typical examples include Eudragit® RS30D which is a low permeability ammonium methacrylate polymer and Eudragit® RL30D which is a high permeability ammonium methacrylate polymer. Eudragit RL and Eudragit RS are water swellable, and the amount of water absorbed by these polymers is pH-dependent, however, dosage forms coated with Eudragit RL and RS are pH-independent.

The acrylic coatings may comprise a mixture of two acrylic resin lacquers commercially available from Rohm Pharma under the Trade names Eudragit® RL30D and Eudragit® RS30D, respectively. The Eudragit® RL/RS dispersions of the present invention may be mixed together in any desired ration in order to ultimately obtain a prolonged-release formulation having a desirable dissolution profile.

Other polymers which can be used as a prolonged release coating materials if they are applied at sufficient amounts are, for example, hydrophilic polymers such as hydroxypropylmethylcellulose.

The above mentioned coatings may also be applied in combination. Further it is possible to influence the release properties of a dosage form by increasing the amount of the coating material and thus the thickness of the coating.

In embodiments of the present invention where the coating comprises an aqueous dispersion of a hydrophobic controlled release material, the inclusion of an effective amount of a plasticizer in the aqueous dispersion of hydrophobic material may further improve the physical properties of the prolonged release coating. For example, because ethyl cellulose has a relatively high glass transition temperature and may not form flexible films under normal coating conditions, it can be preferred to incorporate a plasticizer into an ethyl cellulose coating containing prolonged release coating before using the same as a coating material. Generally, the amount of plasticizer included in a coating solution is based on the concentration of the film-former, e.g., most often from about 1 to about 50% by weight of the film-former.

Examples of suitable plasticizers for ethyl cellulose include water insoluble plasticizers such as dibutyl sebacate, diethyl phthalate, triethyl citrate, tributyl citrate, and triacetin, although it is possible that other water-insoluble plasticizers (such as acetylated monoglycerides, phthalate esters, castor oil, etc.) may be used. Triethyl citrate is an especially preferred plasticizer for the aqueous dispersions of ethyl cellulose of the present invention.

Examples of suitable plasticizers for the acrylic polymers of the present invention include, but are not limited to citric acid esters such as triethyl citrate NF XVI, tributyl citrate, dibutyl phthalate, and possibly 1,2-propylene glycol. Other plasticizers which have proved to be suitable for enhancing the elasticity of the films formed from acrylic films such as Eudragit® RL/RS lacquer solutions include polyethylene glycols, propylene glycol, diethyl phthalate, castor oil and triacetin.

The pharmaceutical compositions in accordance with the invention as described herein may be formulated to provide a mean AUCt of about 1162 h*pg/ml to about 2241 h*pg/ml and preferably of about 1328 to about 2075 h*pg/ml per mg administered amount of hydromorphone and a mean Cmax of about 122 pg/ml to about 234 pg/ml and preferably of about 139 to about 218 pg/ml per mg administered amount of hydromorphone and mean tmax of about 1 h to about 4.5 h, preferably of about 1.5 h to about 4 h and more preferably of about 1.5 h to about 3 h. These values refer preferably to single dose administration to healthy subjects. Preferably, administration is in the fasted state. The mean values of Cmax, AUCt and tmax refer to the geometric mean.

The pharmaceutical compositions in accordance with the invention as described herein (particularly the coated bead embodiment) may be formulated to provide a mean AUCt of about 5.900 ng*h/mL to about 8.400 ng*h/mL and preferably of about 6.500 to about 8.400 ng*hg/mL per mg administered amount of hydromorphone and a mean Cmax of about 0.390 ng/ml to about 0.726 ng/mL and preferably of about 0.590 to about 0.726 ng/mL per mg administered amount of hydromorphone and mean tmax of about 1 h to about 4.5 h, preferably of about 1.5 h to about 4 h and more preferably of about 4.0 h to about 6.5 h. These values refer preferably to single dose administration to healthy subjects. Preferably, administration is in the fasted state. The mean values of Cmax, AUCt and tmax refer to the geometric mean.

The “Cmax value” indicates the maximum blood plasma concentration of the active agent hydromorphone.

The “tmax value” indicates the time point at which the Cmax value is reached. In other words, tmax is the time point of the maximum observed plasma concentration.

The “AUC (Area Under the Curve)” value corresponds to the area of the concentration curve. The AUC value is proportional to the amount of the active agent absorbed into the blood circulation in total and is hence a measure for the bioavailability.

The “AUCt value” is the value for the area under the plasma concentration-time curve from the time of administration to the last measurable concentration. AUCt values are usually calculated using the linear trapezoidal method.

If pharmacokinetic parameters such as mean tₘₐₓ, cₘₐₓ and AUCt are measured for healthy subjects which may be healthy human, they are typically obtained by measuring the development of blood plasma values over time in a test population of approximately 16 to 24 healthy human subjects. Regulatory bodies such as the European Agency for the Evaluation of Medicinal Products (EMEA) or the Food and Drug Administration (FDA) will usually accept data obtained from e.g. 16 or 24 test persons. However, initial trials involving fewer participants such as 8 to 16 participants may also be acceptable.

The term “healthy” subjects in this context refers to a typical male or female of usually Caucasian origin with average values as regards height, weight and physiological parameters such as blood pressure etc. Healthy human subjects for the purposes of the present invention are selected according to inclusion and exclusion criteria which are based on and in accordance with recommendations of the International Conference for Harmonization of Clinical Trials (ICH).

For the purposes of the present invention, healthy subjects may be identified according to conventional inclusion and exclusion criteria.

Thus, inclusion criteria comprise, for example, an age between ≧18 and ≦45 years; a BMI within the range 19-29 kg/m², and within the weight range 60-100 kg for males and 55-90 kg for females; that females must be non-nursing, non-pregnant, and provide a negative urine β-hCG pregnancy test within 24 hours before receiving the study medication; generally good health, evidenced by a lack of significantly abnormal findings on medical history, physical examination, clinical laboratory tests, vital signs, and ECG and the like.

Exclusion criteria comprise, for example, exposure to any investigational drug or placebo within 3 months of the first dose of study medication, any significant illness within the 30 days before the first dose of study medication, any clinically significant abnormalities identified at prestudy screening for medical history, physical examination or laboratory analyses, use of any prescription medication (except HRT for postmenopausal females and contraceptive medication) in the 21 days, or over the counter medication including acid controllers, vitamins, herbal products and/or mineral supplements in the 7 days, before first dose of study medication, concurrent medical condition known to interfere with gastrointestinal drug absorption (e.g. delayed gastric emptying, mal absorption syndromes), distribution (e.g. obesity), metabolism or excretion (e.g. hepatitis, glomerulonephritis), history of or concurrent medical condition, which in the opinion of the investigator would compromise the ability of the subject to safely complete the study, history of seizure disorders for which subjects required pharmacologic treatment, current history of smoking more than 5 cigarettes a day, subjects with evidence of active or past history of substance or alcohol abuse according to DSM-IV criteria, subjects who reported regular consumption of 2 or more alcoholic drinks per day or have blood alcohol levels of ≧0.5% at screening, donation of more than 500 mL of blood or blood products or other major blood loss in the 3 months before first dose of study medication, any positive results in the prestudy screen for ethanol, opiates, barbiturates, amphetamines, cocaine metabolites, methadone, propoxyphene, phencyclidine, benzodiazepines, and cannabinoids in the specimen of urine collected at screening, known sensitivity to hydromorphone, naloxone, or related compounds and the like.

The pharmaceutically acceptable excipients may include the fillers, binders, lubricants, release rate modifiers, spheronising agents, anti-tacking agents, etc. as mentioned above. However, some of these excipients such as, for example, lubricants may be added at a later stage.

Different technology is available to obtain such granules. One may use, for example, drum granulation or fluidized bed granulation.

The granules which may be produced by wet granulation extrusion may be dried before being mixed with the at least one pharmaceutically active agent.

Typically, drying takes place at humidity in the range of about 0.5% to about 5.0% at a temperature in the range of about 20° C. to about 90° C. and for a time in the range of about 10 min to about 3 hours. Drying at ambient humidity at a temperature in the range of about 40° C. to about 90° C. and for a time in the range of about 15 min to about 2 hours can be preferred.

The granules may then be optionally screened in order to select granules of substantially uniform size. Selecting granules of substantially uniform size before compressing them may improve the prolonged release properties of the final prolonged release pharmaceutical composition as the active and the granules are then assumed to be more uniformly distributed which may prevent irregularities in the release profile. Granules for which at least about 70%, preferably at least about 80%, more preferably at least about 90% are of about the same mean size will typically be considered as being of substantially uniform size.

Preferably, granules are selected of a mean size in the range of about 100 μm to about 2 mm, more preferably in the range of about 100 μm to about 1 mm, and even more preferably in the range of about 100 μm to about 600 μm. Selection may be performed using a sieve with an appropriate mesh size.

In some embodiments the granules may be milled before selecting them for their size. Milling may both increase the yield of the selection step and improve the granules' suitability for the subsequent compression step. For milling one may use for example a rotary hammer mill or top/bottom driven conical mill.

For compressing the pharmaceutically active agent(s) with the granules, one may use typical tabletting equipment such as Example Fette or Kilian press.

When compressing granules and active(s), one may also include pharmaceutically acceptable excipients as they are commonly used in the art. For example, one may add lubricants, anti-tacking agents, binders and the like. For lubricants, the use of magnesium stearate and/or talc in the aforementioned amounts can be of advantage.

As mentioned above, prolonged release pharmaceutical dosage forms in accordance with the invention may be additionally subjected to a heat treatment step as has been described above.

The prolonged release coating may be produced by methods common in the art such a fluidized bed spraying.

Various embodiments of the present application will be illustrated with reference to the following non-limiting examples which should not be used to construe the scope of the invention.

### EXAMPLES

While the embodiments exemplified below are focussed on such a prolonged release dosage form in the form of coated beads, it is believed that the improvement in stability and/or dissolution properties will also be seen in other dosage forms such as those described in Danagher. Thus, it is believed that that the improvement in stability and/or dissolution properties will also be seen in other dosage forms such as matrix dosage forms and the like comprising (i) hydromorphone or a pharmaceutically acceptable salt thereof and (ii) naloxone or a pharmaceutically acceptable salt thereof as the active ingredients. Therefore, the invention is intended to cover these additional prolonged release dosage forms.

The controlled release bead dosage forms from Formulation A and Formulation B in Example 18 of Danagher have been further improved to meet stability requirements on related substances and dissolution release rate within product shelf life. It was found that inclusion of the combination of an oxidizing agent (such as sodium metabisulfate) and a chelating agent (such as sodium EDTA) to Formulations A and B in Example 18 of Danager resulted in an improvement in the total impurities at the 24 months proposed shelf life—i.e., an improvement in the stability of the formulation.

A number of variables were studied and based on the studies excipients were identified and adjusted to obtain a finished product that is stable within the product shelf life. The general method of manufacture is described below followed by the various studies and findings.

The controlled-release multiparticulate bead formulation of hydromorphone and naloxone may be conveniently manufactured in three stages: (i) an immediate-release coating (drug layering), a controlled-release coating, and (iii) top coating. In the Examples below, all three stages were carried out in a fluid bed dryer with Wurster column.

### Example 1

This Example involved the addition of one or both of an antioxidant (such as sodium metabisulfite) and a chelating agent (such as ethylenedinitrotetraacetic acid disodium salt dihydrate). These were added at the drug layering stage to prevent any degradation of the active pharmaceutical ingredients. Pharmaceutical preparations were produced according to the specifics shown in Table 1. It should be noted that Formulations A and B are same as in Example 18 of Danagher—these Formulations did not contain antioxidant and/or chelating agent.

TABLE 1
Effect of antioxidant and chelating agent on total impurities

| | HN021WF2 | HN1136U | HN1137U | HN021U | Formula A | Formula B |
|---|---|---|---|---|---|---|
| | Amount | Amount | Amount | Amount | Amount | Amount |
| Ingredient | (mg) | (mg | (mg) | (mg) | (mg) | (mg) |
| Hydromorphone hydrochloride | 3.00 | 3.00 | 3.00 | 3.00 | 3.00 | 3.00 |
| Naloxone hydrochloride dihydrate | 1.64 | 1.65 | 1.65 | 1.64 | 1.65 | 1.65 |
| Microcrystalline cellulose (MCC) spheres | 44.97 | 44.84 | 44.84 | 44.97 | 44.89 | 44.83 |
| Hydroxypropyl methylcellulose, | 1.64 | 0.50 | 0.50 | 1.64 | 1.63 | 1.68 |
| polyethylene glycol film coating | | | | | | |
| concentrate | | | | | | |
| Sodium Metabisulfite | 0.02 | 0.02 | — | 0.02 | — | — |
| Sodium EDTA | — | 0.02 | — | — | — | — |
| Aqueous ethylcellulose dispersion | 4.76 | 3.25 | 3.25 | 4.76 | 4.66 | 6.04 |
| Polyvinyl alcohol-polyethylene glycol | 0.28 | 0.25 | 0.25 | 0.28 | 0.34 | 0.45 |
| graft copolymer | | | | | | |
| Purified Water USP | qs | qs | qs | qs | qs | qs |
| Total | 56.31 | 53.78 | 53.74 | 56.31 | 56.17 | 57.65 |
| Hard Gelatin capsule | Size 4 | Size 4 | Size 4 | Size 4 | Size 4 | Size 4 |
| Total filled capsule weight | 92.15 | 93.99 | 94.30 | 92.15 | 92.01 | 93.49 |
| % Total impurities 25° C./60% RH 6 | — | 0.51 | 2.51 | 0.09 | 2.26 | 0.58 |
| months with desiccant | | | | | | |
| % Total impurities 25° C./60% RH 6 | 0.57 | — | — | 0.66 | — | 2.55 |
| months without desiccant | | | | | | |
| % Total impurities 25° C./60% RH 12 | 1.65 | — | — | 2.12 | 3.09 | 2.28 |
| months without desiccant | | | | | | |
| Total impurities 25° C./60% RH 12 months | 0.45 | — | — | — | — | |
| with desiccant | | | | | | |

It can be concluded from the stability data that the addition of sodium metabisulfite and ethylenedinitrotetraacetic acid disodium salt dihydrate improved the stability of hydromorphone HCl and naloxone HCl in the finished product. Based on these results, it is believed that the use of an antioxidant such as sodium metabisulfite and a chelating agent such as ethylenedinitrotetraacetic acid disodium salt dihydrate to the drug layer result improvement in stability of the formulation.

### Example 2

This Example was focussed on the core substrate. Specifically, during the drug layering process, an aqueous solution was prepared by mixing hydromorphone HCl/naloxone HCl with a binder (such as hydroxypropyl methylcellulose, polyethylene glycol film coating concentrate or polyvinyl alcohol-polyethylene glycol graft copolymer) together with sodium metabisulfite and ethylenedinitrotetraacetic acid disodium salt dihydrate. This solution was sprayed onto the core substrate.

The core substrate type was varied to understand the impact on the degradation stability profiles of the finished product. Initial assessments suggested sugar spheres could present incompatibilities with hydromorphone HCl and naloxone HCl. Therefore, four types of substrate were then selected to produce formulations based on the specifics set on in Table 2:
(1) microcrystalline Cellulose Spheres (MCC spheres, Cellets® 700),
(2) microcrystalline Cellulose Spheres (MCC spheres, Cellets® 700) pre-coated with about 20% polyvinylalcohol-polyvinylalcohol-polyethylene glycol copolymer (Kollicoat Protect-moisture barrier excipient), described as KPM spheres,
(3) silica spheres, and
(4) mannitol-polyvinylpyrrolidone spheres (18/20 mesh).

TABLE 2
Effect of core substrate on total impurities

| | HN1213PU | HN1216KU | HN1223KU | HN1227KU |
|---|---|---|---|---|
| Ingredient | Amount (mg) | Amount (mg) | Amount (mg) | Amount (mg) |
| Hydromorphone hydrochloride | 3.00 | 3.00 | 3.00 | 3.00 |
| Naloxone hydrochloride dihydrate | 1.65 | 1.65 | 1.65 | 1.65 |
| Microcrystalline cellulose (MCC) | 37.20 | 44.74 | — | — |
| spheres | | | | |
| Mannitol-Polyvinylpyrrolidone spheres | — | — | 44.74 | — |
| Silica spheres | — | — | — | 44.74 |
| Sodium Metabisulfite | 0.05 | 0.05 | 0.05 | 0.05 |
| Sodium EDTA | 0.05 | 0.05 | 0.05 | 0.05 |
| Aqueous ethylcellulose dispersion | 4.65 | 4.66 | 4.66 | 4.66 |
| Polyvinyl alcohol-polyethylene glycol | 0.85 | 2.55 | 2.55 | 2.55 |
| graft copolymer | | | | |
| Polyvinyl alcohol-Polyvinyl alcohol- | 18.50 | — | — | — |
| polyethylene glycol copolymer | | | | |
| (Kollicoat Protect) | | | | |
| Q7-2587, 30% Simethicone emulsion* | 0.13 | — | — | — |
| Purified Water USP | qs | qs | qs | qs |
| Total | 66.08 | 56.70 | 56.70 | 56.70 |
| Hydroxypropyl methylcellulose | Size 4 | Size 4 | Size 4 | — |
| capsule | | | | |
| Total filled capsule weight | 106.62 | 96.47 | 97.61 | — |
| % Total impurities 60° C./95% RH with | 2.57 | 4.75 | 2.84 | 4.03 |
| desiccant performed on beads only | 168 hours | 168 hours | 168 hours | 168 hours |
| % Total impurities 40° C./75% RH 1 | 0.60 | 0.30 | 0.40 | — |
| month with desiccant on capsules | | | | |

*Added to Kollicoat Protect

The stability data indicated the starting core substrate material has a noticeable effect on the amount of total impurities in the finished product. Based on the accelerated stability data, it is evident that the mannitol-polyvinylpyrrolidone spheres and KPM sphere are more effective in providing a more stable finished product in terms of controlling the formation of degradation products. Furthermore, it was also established that the mannitol-polyvinylpyrrolidone spheres are more effective controlling unknown degradation products, whereas KPM spheres are more effective in controlling known degradation products than MCC spheres. However, mannitol-polyvinylpyrrolidone and microcrystalline cellulose uncoated spheres are more effective in controlling the formation of unknown degradation product within the finished product. Therefore, mannitol-polyvinylpyrrolidone and microcrystalline cellulose uncoated spheres are believed to be superior overall for use in the present prolonged release dosage form.

As a result, during the drug layering process, an aqueous solution is prepared by mixing hydromorphone HCl, naloxone HCl, sodium metabisulfite, ethylenedinitrotetraacetic acid disodium salt dihydrate and hydroxypropyl methylcellulose, polyethylene glycol film coating concentrate or polyvinyl alcohol-polyethylene glycol graft copolymerin water. The clear solution is then sprayed on to microcrystalline cellulose spheres (MCC spheres) or mannitol-polyvinylpyrrolidone spheres to manufacture Immediate Release (IR) beads.

The controlled-release (CR) beads (also referred to throughout this specification as prolonged release) are produced by coating the IR beads with a dispersion of aqueous ethylcellulose dispersion and a pore former such as polyethylene glycol film coating concentrate. The amount of this controlled release suspension is optimised depending on the equipment and manufacturing batch size by applying several different ratios of aqueous ethylcellulose dispersion to polyethylene glycol film coating concentrate; ranging from 80:20 to 97:3. The percentage weight gain (8% to 17%) was also varied to effectively control the release rate and obtain the targeted dissolution profile as per formulations A and B in B in Example 18 of Danagher.

### Example 3

This Example was focussed on illustrating the use of a top coat on the controlled release beads to achieve desired dissolution and total impurities amount, and if needed, the effect of the polymer used in the top coating process stage on the stability and dissolution rate of the product. Table 3 sets out the specifics of the formulations produced in this Example.

TABLE 3
Effect of top coat on dissolution release rate over time

| | Formula A | HN1104U | HN1107BU | HN1125KU | HN1212U | HN1213PU |
|---|---|---|---|---|---|---|
| | Amount | Amount | Amount | Amount | Amount | Amount |
| Ingredient | (mg) | (mg) | (mg | (mg) | (mg) | (mg) |
| Hydromorphone hydrochloride | 3.00 | 3.00 | 3.00 | 3.00 | 3.00 | 3.00 |
| Naloxone hydrochloride dihydrate | 1.65 | 1.64 | 1.65 | 1.64 | 1.65 | 1.65 |
| Microcrystalline cellulose (MCC) spheres | 44.89 | 44.97 | 44.97 | 44.89 | 37.30 | 37.20 |
| Hydroxypropyl methylcellulose, | 0.50 | 0.50 | 0.50 | — | — | — |
| polyethylene glycol film coating | | | | | | |
| concentrate | | | | | | |
| Sodium Metabisulfite | — | — | — | — | 0.05 | 0.05 |
| Sodium EDTA | — | — | — | — | 0.05 | 0.05 |
| Aqueous ethylcellulose dispersion | 4.66 | 6.39 | 6.39 | 4.66 | 4.66 | 4.65 |
| Polyvinyl alcohol-polyethylene glycol graft | 0.34 | 1.07 | 1.07 | 0.85 | 0.85 | 0.85 |
| copolymer | | | | | | |
| Hydroxypropyl methylcellulose, | 1.13 | — | — | 1.13 | — | — |
| polyethylene glycol film coating | | | | | | |
| concentrate top coat | | | | | | |
| Q7-2587,30% Simethicone emulsion* | | | | | 0.05 | 0.13 |
| Opadry AMB top coat 8% weight gain | — | — | 4.60 | — | — | — |
| Polyvinyl alcohol-Polyvinyl alcohol- | — | — | — | — | — | 11.00 of |
| polyethylene glycol copolymer (Kollicoat | | | | | | (18.50) |
| Protect) | | | | | | |
| Opadry 200 top coat 20% weight gain | | — | — | — | — | — |
| Polyvinyl alcohol-polyethylene glycol graft | — | — | — | — | — | — |
| copolymer top coat 2-3% | | | | | | |
| Purified Water USP | qs | qs | qs | qs | qs | qs |
| Total | 56.17 | 57.52 | 62.12 | 56.18 | 55.11 | 66.08 |

*Added to Kollicoat Protect

TABLE 3
Effect of top coat on dissolution release rate over time

| | HN1207 | HN1216KU | PT120027 | PT120028 |
|---|---|---|---|---|
| | Amount | Amount | Amount | Amount |
| Ingredient | (mg) | (mg) | (mg | (mg) |
| Hydromorphone hydrochloride | 3.00 | 3.00 | 3.00 | 3.00 |
| Naloxone hydrochloride dihydrate | 1.65 | 1.65 | 1.65 | 1.65 |
| Microcrystalline cellulose (MCC) spheres | 44.87 | 44.74 | 44.78 | 44.78 |
| Hydroxypropyl methylcellulose, | 0.50 | — | 0.50 | 0.50 |
| polyethylene glycol film coating | | | | |
| concentrate | | | | |
| Sodium Metabisulfite | — | 0.05 | 0.05 | 0.05 |
| Sodium EDTA | — | 0.05 | 0.05 | 0.05 |
| Aqueous ethylcellulose dispersion | 4.66 | 4.66 | 3.72 | 3.72 |
| Polyvinyl alcohol-polyethylene glycol graft | 0.34 | 0.84 | 0.28 | 0.28 |
| copolymer | | | | |
| Hydroxypropyl methylcellulose, | — | — | — | — |
| polyethylene glycol film coating | | | | |
| concentrate top coat | | | | |
| Q7-2587,30% Simethicone emulsion* | | — | — | — |
| Opadry AMB top coat 8% weight gain | — | — | — | — |
| Polyvinyl alcohol-polyethylene glycol graft | — | — | — | — |
| copolymer top coat 20% weight gain | | | | |
| Opadry 200 top coat 20% weight gain | 11.20 | — | | — |
| Polyvinyl alcohol-polyethylene glycol graft | — | 1.71 | — | 1.10 |
| copolymer top coat 2-3% | | | | |
| Purified Water USP | qs | qs | qs | qs |
| Total | 66.05 | 56.70 | 54.03 | 55.13 |

Formula A

| | | | |
|---|---|---|---|
| | Initial | 1 M 30° C./ | 12 M |
| Time | beads | 65% RH beads | RT beads |

| hour | HH | Nal | HH | Nal | HH | Nal |
|---|---|---|---|---|---|---|
| 0 | 0 | 0 | 0 | 0 | 0 | 0 |
| 1 | 3 | 4 | 5 | 6 | 4 | 6 |
| 2 | 6 | 7 | 14 | 15 | 12 | 13 |
| 3 | 11 | 12 | 28 | 28 | 22 | 23 |
| 4 | 20 | 21 | 43 | 43 | 36 | 35 |
| 6 | 45 | 44 | 68 | 68 | 60 | 58 |
| 8 | 67 | 65 | 83 | 82 | 76 | 73 |
| 10 | 79 | 77 | 90 | 90 | 85 | 82 |
| 12 | 87 | 85 | 95 | 95 | 90 | 87 |
| 16 | 96 | 94 | 100 | 101 | 96 | 93 |

HN1104U (CR batch of HN1107BU)

| | | |
|---|---|---|
| | | 1 M 30° C./ |
| Time | Initial beads | 65% RH beads |

| hour | HH | Nal | HH | Nal |
|---|---|---|---|---|
| 0 | 0 | 0 | 0 | 0 |
| 1 | 13 | 13 | 16 | 15 |
| 2 | 32 | 32 | 39 | 38 |
| 3 | 47 | 46 | 56 | 54 |
| 4 | 59 | 57 | 68 | 66 |
| 6 | 75 | 73 | 82 | 80 |
| 8 | 85 | 83 | 89 | 87 |
| 10 | 91 | 89 | 94 | 93 |
| 12 | 95 | 93 | 97 | 96 |
| 16 | 99 | 98 | 100 | 99 |

HN1107BU

| | | |
|---|---|---|
| | Initial | 1 M 30° C./ |
| Time | beads | 65% RH beads |

| hour | HH | Nal | HH | Nal |
|---|---|---|---|---|
| 0 | 0 | 0 | 0 | 0 |
| 1 | 23 | 23 | 29 | 28 |
| 2 | 49 | 48 | 59 | 58 |
| 3 | 66 | 64 | 76 | 74 |
| 4 | 76 | 74 | 86 | 84 |
| 6 | 88 | 86 | 95 | 93 |
| 8 | 94 | 93 | 100 | 99 |
| 10 | 98 | 97 | 102 | 101 |
| 12 | 100 | 99 | 103 | 103 |
| 16 | 102 | 102 | 105 | 105 |

HN1125KU

| | | |
|---|---|---|
| | Initial | 9 M |
| Time | beads | RT beads |

| hour | HH | Nal | HH | Nal |
|---|---|---|---|---|
| 0 | 0 | 0 | 0 | 0 |
| 1 | 1 | 3 | 1 | 2 |
| 2 | 2 | 5 | 3 | 4 |
| 3 | 3 | 6 | 4 | 5 |
| 4 | 5 | 8 | 6 | 7 |
| 6 | 12 | 16 | 16 | 19 |
| 8 | 23 | 27 | 27 | 29 |
| 10 | 35 | 38 | 37 | 39 |
| 12 | 44 | 47 | 45 | 47 |
| 16 | 55 | 59 | 56 | 57 |

| HN1212U (CR batch | | |
|---|---|---|
| of HN1213PU) | | HN1213PU |
| | Initial | Initial |
| Time | beads | beads |

| hour | HH | Nal | HH | Nal |
|---|---|---|---|---|
| 0 | 0 | 0 | 0 | 0 |
| 1 | 3 | 6 | 16 | 18 |
| 2 | 18 | 22 | 66 | 66 |
| 3 | 49 | 52 | 93 | 93 |
| 4 | 70 | 72 | 100 | 100 |
| 6 | 86 | 86 | 101 | 101 |
| 8 | 93 | 93 | 101 | 102 |
| 10 | 96 | 97 | 102 | 102 |
| 12 | 98 | 98 | 102 | 102 |
| 16 | 100 | 100 | 102 | 103 |

HN1216KU

| | | |
|---|---|---|
| | Initial | 3 Months 30° C./ |
| Time | beads | 65% RH |

| hour | HH | Nal | HH | Nal |
|---|---|---|---|---|
| 0 | 0 | 0 | 0 | 0 |
| 1 | 1 | 3 | 3 | 3 |
| 2 | 7 | 9 | 9 | 8 |
| 3 | 18 | 21 | 20 | 19 |
| 4 | 32 | 36 | 33 | 33 |
| 6 | 52 | 55 | 54 | 54 |
| 8 | 64 | 66 | 65 | 65 |
| 10 | 72 | 74 | 73 | 73 |
| 12 | 77 | 79 | 78 | 78 |
| 16 | 84 | 86 | 85 | 84 |

| PT120027 | | | |
|---|---|---|---|
| (CR batch of | | | |
| PT120028) | | PT120028 | PT120028 |
| | Initial | Initial | 1 month |
| Time | beads | beads | caps RT |

| hour | HH | HH | Nal | Nal | HH | Nal |
|---|---|---|---|---|---|---|
| 0 | 0 | 0 | 0 | 0 | 0 | 0 |
| 1 | 5 | 7 | 7 | 9 | 5 | 6 |
| 2 | 12 | 14 | 14 | 16 | 13 | 15 |
| 3 | 19 | 21 | 23 | 25 | 22 | 24 |
| 4 | 28 | 29 | 31 | 33 | 32 | 33 |
| 6 | 45 | 46 | 47 | 48 | 51 | 51 |
| 8 | 59 | 59 | 61 | 61 | 66 | 66 |
| 10 | 70 | 69 | 71 | 70 | 76 | 76 |
| 12 | 78 | 76 | 78 | 77 | 83 | 83 |
| 16 | 87 | 85 | 87 | 86 | 92 | 92 |

This results of this Example clear illustrate the beneficial effect of adding a top coat to the controlled release beads. It can be concluded that the addition of the polymer has an improved effect on the stability of the finished product. The addition of the top coat increased the dissolution rate of the beads at the controlled release stage. This increase was less than 5% when polyvinyl alcohol-polyethylene glycol graft copolymer was added in a 2% to 3% weight gain of the controlled release beads. In all other cases whether another polymer was added or not top coat was added on the controlled release beads, the release rate was greater than 5% and continued increasing over time. Moreover, other polymers such as Opadry clear, Opadry AMB, Opadry 200 and Kollicoat Protect that are typically used as moisture control barrier polymers did not offer significant moisture protection as the polyvinyl alcohol-polyethylene glycol graft copolymer did. Dissolution test of batch HN1207 was not performed as the stability data showed a 4.99% total impurities at 60° C./95% RH for 96 hours.

Batch PT120027 and its top coat batch PT120028 clearly showed the variability of the dissolution release profiles at different time points is less than 5%.

Batches PT120028 and HN1216KU, where polyvinyl alcohol-polyethylene glycol graft copolymer was added in a 2% to 3% weight gain during the manufacturing of the top coated beads also showed a dissolution rate variability less than 10% over time.

Based on results in this Example, in the third stage of the manufacturing process, the controlled release beads are more stable when coated with an aqueous solution of polyvinyl alcohol-polyethylene glycol graft copolymer.

Dissolution rate is an important quality attribute of the finished product and, as set out in the results of this Example, it can be controlled with the addition of polyvinyl alcohol-polyethylene glycol graft copolymer as a top coating system. Of note, Formulations A and B in Example 18 of Danagher did not contain this polymer in the top coating layer.

The following equipment process conditions were used in this Example:
Equipment: GPCG1 Wurster coater
Nozzle diameter: 1.0 mm
Atomising pressure: 2 bar
Air velocity: 6-7 m/s

The coating temperatures and spray rates used at different stages are provided in Table 4.

TABLE 4
Process parameters during manufacturing process

| | | | | | | | Top Coat |
|---|---|---|---|---|---|---|---|
| | | | | | | | Polyvinyl |
| | | | Top | | Top | | alcohol- |
| | Drug | Controlled | Coat | Top Coat | Coat | Top Coat | polyethylene |
| | layering | Release | Opadry | Opadry | Opadry | Kollicoat | glycol graft |
| Coating stage | (IR beads) | beads | Clear | 200 | AMB | Protect | copolymer |
| Inlet | 60-65 | 50-55 | 55-60 | 54-61 | 59-65 | 53-54 | 42-44 |
| temperature | | | | | | | |
| (C.) | | | | | | | |
| Product | 45-47 | 40-42 | 45-47 | 44-46 | 50-52 | 44-46 | 35-37 |
| temperature | | | | | | | |
| (C.) | | | | | | | |
| Spray rate | 1.3-6.7 | 4-12.6 | 1.2-8 | 2-8 | 1.5-3.9 | 2-6 | 2-8 |
| ml/min | | | | | | | |

### Example 4

In this Example, a number of pharmaceutical formulations were prepared based on the particulars set out in Table 5.

The bulk beads were encapsulated in hard shell capsules. This can be done using hydroxypropyl methyl cellulose (also referred to as hypromellose) or hard gelatin capsules. The focus of this study was to determine the effect of the capsule shell type on the stability of the product.

TABLE 5
Effect of capsule shell type on total impurities

| | HN1136U | HN1136U |
|---|---|---|
| | Amount | Amount |
| Ingredient | (mg) | (mg) |
| Hydromorphone hydrochloride | 3.00 | 3.00 |
| Naloxone hydrochloride dihydrate | 1.65 | 1.65 |
| Microcrystalline cellulose (MCC) spheres | 44.84 | 44.84 |
| Hydroxypropyl methylcellulose, | 0.50 | 0.50 |
| polyethylene glycol film coating concentrate | | |
| Sodium Metabisulfite | 0.02 | 0.02 |
| Sodium EDTA | 0.02 | 0.02 |
| Aqueous ethylcellulose dispersion | 3.25 | 3.25 |
| Polyvinyl alcohol-polyethylene glycol graft | 0.25 | 0.25 |
| Silicon dioxide NF (Syloid 244FP) NF | 0.25 | 0.25 |
| Purified Water USP | qs | qs |
| Total | 53.78 | 53.78 |
| Hydroxypropyl methylcellulose capsule | — | Size 4 |
| Hard Gelatin capsule | Size 4 | — |
| Total filled capsule weight | 93.99 | 93.99 |
| Desiccant type | Minipax 1 g | Minipax 1 g |
| % Total impurities initial | 0.16 | 0.15 |
| % Total impurities RT3 month | 0.17 | 0.10 |
| % Total impurities 25° C./60% RH 3 month | 0.12 | 0.16 |
| % Total impurities 25° C./60% RH 6 month | 0.51 | 0.36 |
| % Total impurities 40° C./75% RH 1 month | 6.26 | 0.27 |

Both hard gelatin capsules and hypromellose capsules were used to encapsulate the final bulk product (top coated beads) and stability studies were conducted concomitantly. The stability data showed a noticeable reduction in the level of degradation products in hypromellose capsules. The results in this study demonstrate that is beneficial to use hypromellose capsules instead of hard gelatin capsules to produce finished product that will meet total impurities specifications within the shelf life of the product. Of note, formulations A and B in Example 18 of Danagher were encapsulated in hard gelatin capsules instead of hypromellose capsules.

### Testing Methodology

In the above Examples, a number of test results were reported. The following methodology was used in developing the test results.

The dissolution of various formulations was performed using USP basket method at 100 rpm in 900 ml of simulated gastric fluid (without enzyme) at 37° C. The samples were withdrawn at the respective time points and analysed on HPLC using UV detector. The in-vitro release data is indicated as percentage dissolved based on the label content of actives tested.

The impurities of various formulations were determined using gradient HPLC method. The samples were extracted with methanol and water and separated on a reverse phase column using mobile phase consisting of potassium phosphate monobasic buffer and methanol. The actives and impurities were detected with UV detector. The results are reported in % for known degradation products and individual unknown degradation products and total impurities.

While this invention has been described with reference to illustrative embodiments and examples, the description is not intended to be construed in a limiting sense. Thus, various modifications of the illustrative embodiments, as well as other embodiments of the invention, will be apparent to persons skilled in the art upon reference to this description. It is therefore contemplated that the appended claims will cover any such modifications or embodiments.

All publications, patents and patent applications referred to herein are incorporated by reference in their entirety to the same extent as if each individual publication, patent or patent application was specifically and individually indicated to be incorporated by reference in its entirety.

1. A prolonged release pharmaceutical dosage form comprising a plurality of coated beads, each of the coated beads comprising:
(a) a granule;
(b) a first layer coated on the granule, the first layer comprising: (i) hydromorphone or a pharmaceutically acceptable salt thereof, (ii) naloxone or a pharmaceutically acceptable salt thereof, (iii) an antioxidant compound, and (iv) a chelating compound; and
(c) a second layer coated on the first layer, the second layer comprising a prolonged release agent.

2. The prolonged release pharmaceutical dosage form of claim 1, wherein (i) and (ii) are present in a weight ratio of from about 2:1 to about 1:2.

3. The prolonged release pharmaceutical dosage form of claim 1, wherein (i) is a pharmaceutically acceptable salt of hydromorphone.

4. The prolonged release pharmaceutical dosage form of claim 1, wherein (i) is hydromorphone hydrochloride.

5. The prolonged release pharmaceutical dosage form of claim 1, wherein (ii) is a pharmaceutically acceptable salt of naloxone.

6. The prolonged release pharmaceutical dosage form of claim 1, wherein (ii) is naloxone hydrochloride.

7. The prolonged release pharmaceutical dosage form of claim 1, wherein the antioxidant compound comprises sodium metabisulfite.

8. The prolonged release pharmaceutical dosage form of claim 1, wherein the chelating agent comprises ethylenediaminetetraacetic acid.

9. The prolonged release pharmaceutical dosage form of claim 1, wherein the chelating agent comprises ethylenediaminetetraacetic acid disodium salt.

10. The prolonged release pharmaceutical dosage form of claim 1, wherein the prolonged release compound is selected from the group consisting of a hydrophobic polymer, a hydrophilic polymer, a protein-derived material, a gum, a substituted or unsubstituted hydrocarbon, a digestible carbohydrate, a fatty acid, a fatty alcohol, a glyceryl ester of a fatty acid, a natural oil, a synthetic oil, a natural wax, a synthetic wax, and any mixture of two or more of any of these.

11. The prolonged release pharmaceutical dosage form of claim 1, wherein the prolonged release compound is selected from the group consisting of a cellulose ether, an acrylic based polymer, an acrylic based copolymer, a methacrylic based polymer, a methacrylic based copolymer, a fatty alcohol, and any mixture of two or more of any of these.

12. The prolonged release pharmaceutical dosage form of claim 1, wherein the prolonged release compound is selected from the group consisting of a neutral acrylic based polymer, a neutral acrylic based copolymer, a neutral methacrylic based polymer, a neutral methacrylic based copolymer, a hydrophobic cellulose ether, a fatty alcohol, and any mixture of two or more of any of these.

13. The prolonged release pharmaceutical dosage form of claim 1, wherein the prolonged release compound is ethyl cellulose.

14. The prolonged release pharmaceutical dosage form of claim 1, wherein the granule is selected from an uncoated microcrystalline cellulose granule and a mannitol-polyvinylpyrrolidone granule.

15. The prolonged release pharmaceutical dosage form of claim 1, further comprising:
(d) a third layer coated on the second layer, the third layer comprising a moisture barrier agent.

16. The prolonged release pharmaceutical dosage form of claim 15, wherein the moisture barrier agent comprises a polyvinyl alcohol-polyethylene glycol graft copolymer.

17. The prolonged release pharmaceutical dosage form of claim 1 in the form of a capsule.

18. The prolonged release pharmaceutical dosage form of claim 17, wherein the capsule contains the plurality of coated beads.

19. The prolonged release pharmaceutical dosage form of claim 18, wherein the capsule is a hydroxypropyl methyl cellulose capsule.

20. A coated bead comprising:
(a) a granule;
(b) a first layer coated on the granule, the first layer comprising: (i) hydromorphone or a pharmaceutically acceptable salt thereof, (ii) naloxone or a pharmaceutically acceptable salt thereof, (iii) an antioxidant compound, and (iv) a chelating compound; and
(c) a second layer coated on the first layer, the second layer comprising a prolonged release agent.

21. The coated bead of claim 20, wherein (i) and (ii) are present in a weight ratio of from about 2:1 to about 1:2.

22. The coated bead of claim 20, wherein (i) is a pharmaceutically acceptable salt of hydromorphone.

23. The coated bead of claim 20, wherein (i) is hydromorphone hydrochloride.

24. The coated bead of claim 20, wherein (ii) is a pharmaceutically acceptable salt of naloxone.

25. The coated bead of claim 20, wherein (i) iii) is naloxone hydrochloride.

26. The coated bead of claim 20, wherein the antioxidant compound comprises sodium metabisulfite.

27. The coated bead of claim 20, wherein the chelating agent comprises ethylenediaminetetraacetic acid.

28. The coated bead of claim 20, wherein the chelating agent comprises ethylenediaminetetraacetic acid disodium salt.

29. The coated bead of claim 20, wherein the prolonged release compound is selected from the group consisting of a hydrophobic polymer, a hydrophilic polymer, a protein-derived material, a gum, a substituted or unsubstituted hydrocarbon, a digestible carbohydrate, a fatty acid, a fatty alcohol, a glyceryl ester of a fatty acid, a natural oil, a synthetic oil, a natural wax, a synthetic wax, and any mixture of two or more of any of these.

30. The coated bead of claim 20, wherein the prolonged release compound is selected from the group consisting of a cellulose ether, an acrylic based polymer, an acrylic based copolymer, a methacrylic based polymer, a methacrylic based copolymer, a fatty alcohol, and any mixture of two or more of any of these.

31. The coated bead of claim 20, wherein the prolonged release compound is selected from the group consisting of a neutral acrylic based polymer, a neutral acrylic based copolymer, a neutral methacrylic based polymer, a neutral methacrylic based copolymer, a hydrophobic cellulose ether, a fatty alcohol, and any mixture of two or more or of any of these.

32. The coated bead of claim 20, wherein the prolonged release compound is ethyl cellulose.

33. The coated bead of claim 20, wherein the granule is selected from an uncoated microcrystalline cellulose granule and a mannitol-polyvinylpyrrolidone granule.

34. The coated bead of claim 20, further comprising:
(d) a third layer coated on the second layer, the third layer comprising a moisture barrier agent.

35. The coated bead of claim 34, wherein the moisture barrier agent comprises a polyvinyl alcohol-polyethylene glycol graft copolymer.

36. The prolonged release pharmaceutical dosage form of claim 1 wherein
the plurality of coated beads are disposed in a hydroxypropyl methyl cellulose capsule
(i) is hydromorphone hydrochloride, (ii) is naloxone hydrochloride, and (i) and (ii) are present in a weight ratio of about 2:1;
the second layer comprises ethyl cellulose; and further comprising
(d) a third layer coated on the second layer, the third layer comprising a polyvinyl alcohol-polyethylene glycol graft copolymer.

37. The prolonged release pharmaceutical dosage form of claim 36, wherein the granule is an uncoated microcrystalline cellulose granule.

38. The prolonged release pharmaceutical dosage form of claim 36, wherein the granule is a mannitol-polyvinylpyrrolidone granule.

39. A method for increasing the stability or dissolution of a prolonged release dosage form comprising (i) hydromorphone or a pharmaceutically acceptable salt thereof and (ii) naloxone or a pharmaceutically acceptable salt thereof, the method comprising incorporating a combination of an antioxidant and a chelating agent into the dosage form.

40-51. (canceled)
